(19) United States
(12) Patent Application Publication
(10) Pub. No.: US 2025/0032013 A1
(43) Pub. Date: Jan. 30, 2025

(54) CONTROL OF A LIGHT SOURCE OF A PULSE OXIMETER

(71) Applicant: Loewenstein Medical Technology S.A., Luxembourg (LU)

(72) Inventor: Farzad SAMIE, Karlsruhe (DE)

(21) Appl. No.: 18/775,216

(22) Filed: Jul. 17, 2024

(30) Foreign Application Priority Data
Jul. 27, 2023 (DE) 102023119940.4

(51) Int. Cl. A61B 5/1455

(52) U.S. Cl. CPC A61B 5/14552; A61B 2562/00

(57) ABSTRACT

A method for controlling a light source of a pulse oximeter comprises receiving a first input value indicating an electrical variable in relation to the light source, the electrical variable comprising an electrical current flowing through the light source and/or an electrical voltage present at the light source; receiving a second input value indicating an amplitude of a sensor signal generated by a light sensor; determining at least one limit value for the electrical variable using the second input value, the at least one limit value being assigned to the minimum or maximum target value of a target value range in which the amplitude is to lie; determining at least one deviation value (Δ) indicating a deviation of the first input value from the at least one limit value; generating a control signal for controlling the light source using the at least one deviation value, such that the amplitude approximates the target value range.

### CROSS-REFERENCE TO RELATED APPLICATIONS

The present application claims priority under 35 U.S.C. § 119 of German Patent Application No. 102023119940.4, filed Jul. 27, 2023, the entire disclosure of which is expressly incorporated by reference herein.

### BACKGROUND OF THE INVENTION

### 1. Field of the Invention

The invention relates to a method for controlling a light source of a pulse oximeter. The invention further relates to a control unit, a computer program and a computer-readable medium for carrying out the method and to a pulse oximeter.

### 2. Discussion of Background Information

A pulse oximeter is generally a device for noninvasive determination of arterial oxygen saturation by measurement of light absorption or light remission when blood-perfused body tissue is transilluminated. Such a pulse oximeter can also be used for monitoring pulse rate.

Pulse oximeters are increasingly being used in portable, battery-powered applications. For example, a pulse oximeter can be attached to a patient during emergency transport and remain with said patient during transfer between different hospital departments. Furthermore, pulse oximeters can be used as plug-in modules for multiparameter patient monitors with a limited energy budget. Such applications mean that there is an increasing demand for pulse oximeters with a relatively low power consumption.

In view of the foregoing it would be advantageous to have available a method by which the power consumption of a pulse oximeter, in particular a battery-operated pulse oximeter, can be reduced without appreciable losses in performance. It would further be advantageous to have available a control unit, a computer program and a computer-readable medium for carrying out such a method and a corresponding pulse oximeter.

### SUMMARY OF THE INVENTION

In a first aspect, the invention provides a method for controlling an (electrical) light source of a pulse oximeter. The pulse oximeter further comprises a light sensor configured to convert a light component transmitted and/or reflected by a body part on irradiation with light from the light source into an (electrical) sensor signal. The method comprises: receiving a first input value indicating an electrical variable in relation to the light source, the electrical variable comprising an electrical current flowing through the light source and/or an electrical voltage present at the light source; receiving a second input value indicating an amplitude of the sensor signal; determining at least one limit value for the electrical variable using the second input value, the at least one limit value being assigned to the minimum or maximum target value of a target value range in which the amplitude is to lie; determining at least one deviation value indicating a deviation of the first input value from the at least one limit value; generating a control signal for controlling the light source using the at least one deviation value, such that the amplitude approximates the target value range.

The method allows saving of power in operation of the pulse oximeter, for example by reducing the current flowing through the light source, referred to as light-source current hereinafter, so long as the quality of the photoplethysmogram (PPG for short) from the sensor signal is still sufficient and the necessary accuracy is still maintained, but increasing said current if relatively strong noise occurs or if the quality of the sensor signal generally becomes worse.

In order to be able to ensure a sufficient quality when determining oxygen saturation, the pulsatile component of the sensor signal in the PPG, also referred to as AC component, should not fall below a specific value.

In general, the amplitude of the sensor signal is approximately linear to the light-source current in every patient. This is because physiological factors influencing the absorption of light by blood-perfused body tissue, such as finger thickness or skin color, do not change significantly during a measurement.

The target value can be reached by adjusting the light-source current appropriately during operation. However, a target value range for the amplitude should be defined in order to avoid excessively large and/or excessively frequent adjustments. So long as the AC component is in the target value range, there is no need to adjust the light-source current. If the AC component is below the minimum target value of the target value range, the light-source current is increased accordingly. By contrast, if the AC component is above than the maximum target value of the target value range, the light-source current is reduced accordingly.

The method can be computer-implemented.

“Sensor signal” can be understood to mean an analog or digital signal.

“Amplitude” can be understood to mean, in particular, an amplitude of the AC component of the sensor signal.

“Electrical variable” can also be understood to mean an electrical variable based on the current and/or the voltage and/or an electrical variable influencing the current and/or the voltage. For example, the electrical variable can also be at least one of the following variables: an adjustable series resistance upstream of the light source; a frequency at which the light source is switched on and switched off; a time ratio between switch-on phases, in which the light source is switched on, and switch-off phases, in which the light source is switched off. For example, the frequency can be a clock frequency and/or the time ratio can be a duty cycle in the context of a pulse width modulation of the light source.

“Target value” as in “minimum target value” or “maximum target value” can be understood to mean a predetermined constant. However, it is also possible that the minimum or maximum target value is varied during operation or that both target values are varied during operation, for example depending on a (measured or estimated) change in specific physiological factors of the particular patient.

In a second aspect the invention provides a control unit. The control unit comprises elements configured to carry out the method described above and below.

The elements can comprise hardware and/or software modules. In particular, the elements can comprise a processor configured to carry out the (computer-implemented) method. In addition, the elements can comprise a memory and/or a data communication interface for wireless and/or wired data communication with peripheral devices, for example a smartphone, a smartwatch, a tablet, a laptop, a PC or a ventilator. In the alternative, the control unit can be solely implemented as hardware, for example in the form of an ASIC component (ASIC=application-specific integrated circuit) or FPGA component (FPGA=field-programmable gate array).

It should be noted that features of the method described above and below can also be features of the control unit (and vice versa).

In a third aspect the invention provides a pulse oximeter. The pulse oximeter comprises a light source, a light sensor and a control unit, as described above and below. The light sensor is designed to convert a light component transmitted and/or reflected by a body part on irradiation with light from the light source into an (electrical) sensor signal. The pulse oximeter, for example the control unit thereof, can be configured to determine an oxygen saturation (sO₂) and/or a pulse from the sensor signal by absorption spectroscopy.

“Light source” can be understood to mean, for example, a light-emitting diode, a laser diode, an incandescent lamp or a combination of at least two of these examples. In particular, the light source can be designed to emit light in at least two different predetermined wavelength ranges, for example red light, (near) infrared light or green light. Examples of suitable wavelengths and wavelength ranges are 660 nm, 750 nm to 850 nm, and 905 nm to 940 nm.

“Light sensor” can be understood to mean, for example, a photodiode, a photocell, a CMOS sensor (CMOS=complementary metal-oxide-semiconductor), a CCD sensor (CCD=charge-coupled device) or a combination of at least two of these examples.

“Pulse oximeter” can also be understood to mean a CO-oximeter.

The pulse oximeter can be configured, for example, as a clip for attachment to the body part, for example a finger, an earlobe or a wrist.

The pulse oximeter can be designed such that the light source and the light sensor can be arranged on the same side of the body part or on mutually opposite sides of the body part.

Further aspects of the invention are a computer program and to a computer-readable medium on which the computer program is stored.

The computer program comprises commands which cause a processor—for example a processor of the control unit described above and below—to carry out the method described above and below when the processor executes the computer program.

The computer-readable medium can be a volatile or non-volatile data memory. For example, the computer-readable medium can be a hard disk, a USB storage device (USB=universal serial bus), a RAM (random-access memory), a ROM (read-only memory), an EPROM (erasable programmable read-only memory), an EEPROM (electrically erasable programmable read-only memory), a flash memory or a combination of at least two of these examples. The computer-readable medium can also be a data communication network which enables program code to be downloaded (e.g. via the Internet), or a cloud.

It should be noted that features of the method described above and below can also be features of the computer program and/or the computer-readable medium (and vice versa).

Various embodiments of the invention are described hereinafter. These embodiments should not be understood as restricting the scope of the invention.

According to one embodiment, the at least one limit value can be determined by multiplying the first input value by a quotient of the minimum or maximum target value and the second input value. In other words, the at least one limit value can change during operation depending on a change in the first input value, i.e., the electrical variable in relation to the light source, and the second input value, i.e., the amplitude of the sensor signal.

According to one embodiment, the at least one deviation value can be determined by subtracting the first input value from the at least one limit value.

According to one embodiment, the at least one limit value can comprise a lower limit value assigned to the minimum target value. In this case, a first deviation value can be determined by subtracting the first input value from the lower limit value. The control signal can then be generated using the first deviation value.

According to one embodiment, the lower limit value L₂_{ₘ} can be determined according to

$${L}_{{2}_{m}} = \frac{t}{{A}_{1}} \times {L}_{1} ,$$

where t is the minimum target value, L₁ is the first input value, and A₁ is the second input value. For example, the lower limit value can be an electrical variable value in which the light source, for example the brightness and/or color thereof, is set such that the amplitude of the sensor signal (approximately) corresponds with the minimum target value.

According to one embodiment, the at least one limit value can comprise an upper limit value assigned to the maximum target value. In this case, a second deviation value can be determined by subtracting the first input value from the upper limit value. The control signal can then be generated using the second deviation value. For example, the upper limit value can be an electrical variable value in which the light source, for example the brightness and/or color thereof, is set such that the amplitude of the sensor signal (approximately) corresponds with the maximum target value.

According to one embodiment, the upper limit value L₂_{_{M}} can be determined according to

$${L}_{{2}_{M}} = \frac{t + ε}{{A}_{1}} \times {L}_{1} ,$$

where t+ε is the maximum target value (in this case, a sum total of the minimum target value t and a predetermined distance ε between the minimum and the maximum target value), L₁ is the first input value, and A₁ is the second input value.

According to one embodiment, the first deviation value and the second deviation value, in particular the signs thereof, can be compared with each other. In this case, the control signal can only be generated or the average value can only be determined if the sign of the first deviation value matches the sign of the second deviation value. Thus, it can be established with low computational cost whether the first input value, for example the current light-source current, is within or outside a limit value range defined by the lower and the upper limit value, and this in turn, because of the (approximately) linear relationship between the current light-source current and the current amplitude of the sensor signal, makes it possible to infer whether the current amplitude is within or outside the target value range. If it is within the target value range, there is no need to adjust the light-source current.

According to one embodiment, an average value can be determined from the first deviation value and the second deviation value. The control signal can then be generated using the average value as the at least one deviation value. The average value can be, for example, an arithmetic mean, geometric mean or root mean square. This can simplify any subsequent computational steps.

According to one embodiment, an adjustment value can be determined using the at least one deviation value and an assignment rule, by means of which possible deviation values are each assigned an adjustment value. In this case, an output value can be determined using the first input value and the adjustment value, in particular by adding the first input value and the (positive or negative) adjustment value. The control signal can then be generated using the output value. “Assignment rule” can be understood to mean, for example, a mathematical function or a lookup table. The assignment rule can be stored, for example, in a memory of the control unit described above and below.

According to one embodiment, the assignment rule can be a sigmoid function or be based on a sigmoid function. “Sigmoid function” can be generally understood to mean a function with an S-shaped graph. The sigmoid function can comprise a linear or approximately linear section. Such a section may be relevant, inter alia, to the stability of the method.

According to one embodiment, the assignment rule can be defined as follows:

$$f ( Δ ) = \frac{2 \times S}{1 + {2}^{- Δ}} - S ,$$

where f(Δ) is the adjustment value, Δ is the at least one deviation value, and S is a maximum permissible magnitude (that is variable or constant in operation of the pulse oximeter) of the adjustment value or a step size.

According to one embodiment, an approximation P for the term 2^{-Δ} based on a series expansion, preferably a Taylor series and particularly preferably a Maclaurin series, can be determined. In this case, the assignment rule can be defined as follows:

$$f ( Δ ) = \frac{2 \times S}{1 + P} - S .$$

This allows improved computational efficiency compared to embodiments where the term 2^{-Δ} is calculated instead of P. Thus, the power consumption of the pulse oximeter can be reduced further. Moreover, this facilitates the implementation of the method as hardware and/or software.

According to one embodiment, the approximation P can be defined as follows:

$$if - Δ \geq 0 , then P = 1 + {\sum }_{n = 1}^{N} \frac{{( k \times ( - Δ ) )}^{n}}{n !}$$

$$and / or$$

$$if - Δ < 0 , then P = \frac{1}{1 + {\sum }_{n = 1}^{N} \frac{{( - k \times ( - Δ ) )}^{n}}{n !}} .$$

Here, N is a predetermined order of the series expansion and k is a predetermined factor. For example, N can be a natural number from 1 to 10, preferably 5, and/or k can be a percentage between 0 and 1, preferably from 0.5 and 1.0 and particularly preferably 0.75.

This allows an approximation that is particularly computationally efficient and/or particularly simple to implement, without noticeable impairment of the accuracy of the method.

According to one embodiment, a distance between the minimum and the maximum target value can be up to about 10 nanoamperes, preferably up to about 5 nanoamperes and particularly preferably up to about 1 nanoampere.

According to one embodiment, the minimum target value can be from about 1 to about 5 nanoamperes, preferably from about 1 to about 3 nanoamperes and particularly preferably about 2 nanoamperes.

With such current values, a good compromise could be achieved in practice between accuracy and energy efficiency.

The light-source current can be theoretically in a range from about 3 mA to about 50 mA. In practice, the light-source current can also assume values outside this range, depending on the particular patient.

According to one embodiment, the pulse oximeter can further comprise a battery for supplying power to at least one electrical or electronic component of the pulse oximeter, in particular the light source, or the entire pulse oximeter. This enables the pulse oximeter to also be worn on the go.

According to one embodiment, the pulse oximeter can further comprise a display unit for displaying at least one value determined using the sensor signal, for example an oxygen saturation or a pulse.

### BRIEF DESCRIPTION OF THE DRAWINGS

Embodiments of the invention are described hereinafter with reference to the accompanying drawings. Neither the description nor the drawings should be understood as restricting the scope of the invention. In the drawings:

FIG. 1 shows a pulse oximeter according to one embodiment of the invention.

FIG. 2 shows a diagram illustrating a target value range for use in a method according to one embodiment of the invention.

FIG. 3 shows a diagram illustrating an assignment rule for use in a method according to one embodiment of the invention.

The figures are purely schematic and not true to scale. If identical reference signs are used in different drawings, then these reference signs designate identical or identically acting features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

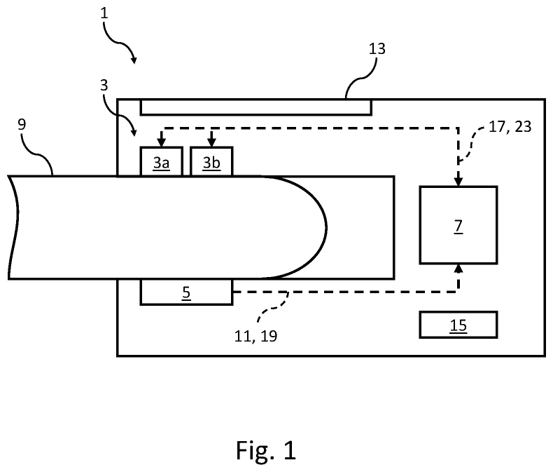

FIG. 1 shows a pulse oximeter 1 comprising an (electrical) light source 3, a light sensor 5 and a control unit 7. The light sensor 5 is designed to convert a light component transmitted and/or reflected by a body part 9, for example a finger, an earlobe or a similarly thin body part, on irradiation with light from the light source 3 into an (electrical) sensor signal 11.

In this example, the control unit 7 is designed to determine an oxygen saturation (sO₂) and/or a pulse from the sensor signal 11.

In addition, the pulse oximeter 1 can comprise a display unit 13 for displaying at least one value and/or at least one graph in relation to the sensor signal 11, in particular in relation to the oxygen saturation and/or the pulse. The display unit 13 can be arranged, for example, in and/or on a housing of the pulse oximeter 1.

It is also possible that the pulse oximeter 1 comprises a battery 15 for supplying power to the pulse oximeter 1. The pulse oximeter 1 can thus also be worn on the go.

In this example, the light source 3 comprises a first light-emitting diode 3a for emitting light in a first wavelength range, for example 660 nm, and a second light-emitting diode 3b for emitting light in a second wavelength range different from the first wavelength range, for example from 905 nm to 940 nm. The control unit 7 can be designed to switch on and switch off the light-emitting diodes 3a, 3b mutually alternatingly in operation of the pulse oximeter 1. The light source 3 can also comprise just one light-emitting diode having a suitably variable wavelength range. Other types of light source such as laser diodes or incandescent lamps are also possible.

The light sensor 5 can comprise, for example, a photodiode, a photocell, a CMOS sensor, a CCD sensor or a combination of at least two of these examples.

The pulse oximeter 1 can be designed, for example, as a clip for attachment to the body part 9 and/or as a CO-oximeter.

In this example, the pulse oximeter 1 is designed such that the light source 3 and the light sensor 5 are arranged on mutually opposite sides of the body part 9 in operation of the pulse oximeter 1. The light sensor 5 thus mainly receives the light component transmitted by the body part 9 when the light source 3 illuminates the body part 9.

In the alternative, the pulse oximeter 1 can be designed such that the light source 3 and the light sensor 5 are arranged on the same side of the body part 9 in operation of the pulse oximeter 1.

The control unit 7 comprises means configured to carry out a specific method for controlling supply of power to the light source 3, as will be described in greater detail below. The means can comprise hardware and/or software modules. In particular, the means can comprise a memory and a processor. A computer program can be stored in the memory, and the processor can be configured to carry out the method by executing the computer program. In addition, the means can comprise a data communication interface for wireless and/or wired data communication with peripheral devices, for example a smartphone, a smartwatch, a tablet, a laptop, a PC or a ventilator.

The control unit 7 can also be solely implemented as hardware, for example in the form of an ASIC component or FPGA component.

The method comprises the following steps.

A first step comprises receiving in the control unit 7, for example in a corresponding hardware and/or software module of the control unit 7, a first input value 17 indicating an electrical variable in relation to the light source 3 and a second input value 19 indicating a current amplitude of the sensor signal 11. The two input values 17, 19 can be measured and/or estimated (i.e., calculated) values. The electrical variable comprises an electrical current currently flowing through the light source 3 and/or an electrical voltage currently present at the light source 3. The electrical variable can also be an electrical variable based on the current and/or the voltage and/or an electrical variable influencing the current and/or the voltage. For example, the electrical variable can also be at least one of the following variables: an adjustable series resistance upstream of the light source 3; a frequency at which the light source 3 is switched on and switched off; a time ratio between switch-on phases, in which the light source 3 is switched on, and switch-off phases, in which the light source 3 is switched off. For example, the frequency can be a clock frequency and/or the time ratio can be a duty cycle in the context of a pulse width modulation of the light source 3. The amplitude can be, in particular, an amplitude of an AC component of the sensor signal 11.

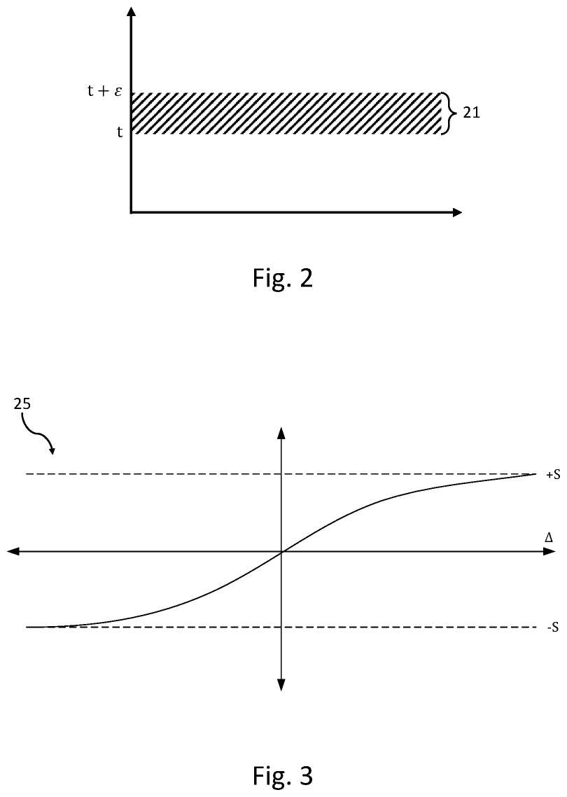

A second step comprises determining at least one limit value for the electrical variable using the second input value 19, i.e., depending on the current amplitude of the sensor signal 11. The at least one limit value is assigned to the minimum target value t or the maximum target value t+ε of a target value range 21 in which the amplitude is to lie (see FIG. 2).

A third step comprises determining at least one deviation value indicating a deviation of the first input value 17 from the at least one limit value.

A fourth step comprises generating a control signal 23 for controlling the light source 3 using the at least one deviation value, such that the amplitude approximates the target value range 21.

The method allows saving of power in operation of the pulse oximeter 1, for example by reducing the current flowing through the light source 3, so long as the quality of the photoplethysmogram (PPG for short) from the sensor signal 11 is still sufficient and the necessary accuracy is still maintained, but increasing said current if relatively strong noise occurs or if the quality of the sensor signal 11 generally becomes worse.

For example, a distance between the minimum target value t and the maximum target value t+ε can be up to 10 nanoamperes, preferably up to 5 nanoamperes and particularly preferably up to 1 nanoampere. In addition or in the alternative, the minimum target value t can be from 1 to 5 nanoamperes, preferably from 1 to 3 nanoamperes and particularly preferably about 2 nanoamperes. Such current values have been found to be particularly suitable in practice in respect of the accuracy achieved and the energy efficiency achieved.

It is possible to determine a lower limit value L₂_{ₘ} and an upper limit value L₂_{_{M}} for the electrical variable. In this case, a first deviation value Δ₁ can be determined by subtracting the first input value 17 from the lower limit value L₂_{ₘ} and a second deviation value Δ₂ can be determined by subtracting the first input value 17 from the upper limit value L₂_{_{M}}. The control signal 23 can then be generated using the two deviation values Δ₁, Δ₂.

For example, the lower limit value L₂_{ₘ} can be determined according to

$${L}_{{2}_{m}} = \frac{t}{{A}_{1}} \times {L}_{1} .$$

In addition or in the alternative, the upper limit value L₂_{_{M}} can be determined according to

$${L}_{{2}_{M}} = \frac{t + ε}{{A}_{1}} \times {L}_{1} .$$

Here, L₁ is the first input value 17, in this case an electrical current flowing through the light-emitting diode 3a and an electrical current flowing through the light-emitting diode 3b, and A₁ is the second input value 19.

Accordingly, the two deviation values Δ₁, Δ₂ are defined as

$${Δ}_{1} = ( \frac{t}{{A}_{1}} - 1 ) \times {L}_{1} and {Δ}_{2} = ( \frac{t + ϵ}{{A}_{1}} - 1 ) \times {L}_{1} .$$

In addition, the two deviation values Δ₁, Δ₂ can be used to determine an average value Δ, for example in the form of an arithmetic mean according to

$$Δ = \frac{{Δ}_{1} + {Δ}_{2}}{2} .$$

The average value Δ can also be a geometric mean or root mean square.

The control signal 23 can then be generated using the average value Δ.

For example, the signs of the two deviation values Δ₁, Δ₂ can be compared with each other. In this case, the average value Δ is determined only if the signs match. Thus, it can be established with low computational cost whether the first input value 17, for example the current light-source current, is within or outside a limit value range defined by the two limit values L₂_{ₘ}, L₂_{_{M}}. This in turn, because of the (approximately) linear relationship between the current light-source current and the current amplitude of the sensor signal 11, makes it possible to infer whether the current amplitude is within or outside the target value range 21. If it is within the target value range 21, there is no need to adjust the light-source current.

It is possible to determine an adjustment value using the average value Δ and an assignment rule 25 (see FIG. 3), by means of which possible deviation values are each assigned an adjustment value. The adjustment value and the first input value 17 can then be used to determine an output value, for example by adding the first input value 17 and the (positive or negative) adjustment value. Lastly, the output value can be used to generate the control signal 23.

The assignment rule 25 can be stored, for example, in the form of a mathematical function or a lookup table in the memory of the control unit 7.

In particular, the assignment rule 25 can be a specific sigmoid function. The sigmoid function can be defined as follows:

$$f ( Δ ) = \frac{2 \times S}{1 + {2}^{- Δ}} - S ,$$

where f(Δ) is the adjustment value and S is a maximum permissible magnitude (that is variable or constant in operation of pulse oximeter 1) of the adjustment value or a step size. This allows avoidance of undesired fluctuations and/or jumps when adjusting the amplitude.

As an option, computational cost, especially when using a microcontroller, can be reduced by calculating an approximation P instead of the term 2^{-Δ} in the sigmoid function, such that it reads:

$$f ( Δ ) = \frac{2 \times S}{1 + P} - S .$$

For example, the approximation is performed according to the following equation:

$${2}^{- Δ} \approx apprx ( - Δ ) = \{ \begin{matrix}1 + {\sum }_{n = 1}^{N} \frac{{( k \times ( - Δ ) )}^{n}}{n !} , & - Δ \geq 0 \\ \frac{1}{1 + {\sum }_{n = 1}^{N} \frac{{( - k \times ( - Δ ) )}^{n}}{n !}} , & - Δ < 0\end{matrix}$$

Here, N is a predetermined order of the series expansion and k is a predetermined factor. For example, N can be a natural number of from 1 to 10, preferably 5, and/or k can be a percentage between 0 and 1, preferably from 0.5 to 1.0 and particularly preferably 0.75. The number N influences the accuracy of approximation and the computational cost. With N=5, a good compromise can be achieved between accuracy and computational cost.

Such a series expansion can also be referred to as a Maclaurin series. This allows an approximation that is particularly computationally efficient and/or particularly simple to implement, without noticeable impairment of the accuracy of the method.

As can be seen in FIG. 3, the assignment rule 25 comprises a relatively long (approximately) linear section around the zero point, which is important for calculation stability.

Finally, it is pointed out that terms such as “have”, “comprise”, “include”, “with”, etc., do not exclude other elements or steps, and indefinite articles such as “a” or “an” do not exclude a plurality.

Furthermore, it is pointed out that features or steps described with reference to one of the embodiments above can also be used in combination with features or steps described with reference to other embodiments from among the embodiments above.

Reference signs in the claims should not be understood as restricting the scope of the subject matter defined by the claims.

### LIST OF REFERENCE SIGNS

1 pulse oximeter
3 light source
3a first light-emitting diode
3b second light-emitting diode
5 light sensor
7 control unit
9 body part
11 sensor signal
13 display unit
15 battery
17 first input value
19 second input value
21 target value range
23 control signal
25 assignment rule
S maximum permissible magnitude of the adjustment value or step size
t minimum target value
t+ε maximum target value
Δ deviation value, average value

What is claimed is:

1. A method for controlling a light source of a pulse oximeter, the pulse oximeter further comprising a light sensor configured to convert a light component transmitted and/or reflected by a body part on irradiation with light from the light source into a sensor signal, wherein the method comprises:
receiving a first input value indicating an electrical variable in relation to the light source, the electrical variable comprising an electrical current flowing through the light source and/or an electrical voltage present at the light source;
receiving a second input value indicating an amplitude of the sensor signal;
determining at least one limit value for the electrical variable using the second input value, the at least one limit value being assigned to a minimum target value (t) or a maximum target value (t+ε) of a target value range in which the amplitude is to lie;
determining at least one deviation value (Δ) indicating a deviation of the first input value from the at least one limit value;
generating a control signal for controlling the light source using the at least one deviation value (Δ), such that the amplitude approximates the target value range.

2. The method of claim 1, wherein the at least one limit value is determined by multiplying the first input value by a quotient of the minimum target value (t) or the maximum target value (t+ε) and the second input value; and/or wherein the at least one deviation value is determined by subtracting the first input value from the at least one limit value.

3. The method of claim 1, wherein the at least one limit value comprises a lower limit value assigned to the minimum target value (t), a first deviation value being determined by subtracting the first input value from the lower limit value, the control signal being generated using the first deviation value; and/or wherein the at least one limit value comprises an upper limit value assigned to the maximum target value (t+ε), a second deviation value being determined by subtracting the first input value from the upper limit value, the control signal being generated using the second deviation value.

4. The method of claim 3, wherein the first deviation value and the second deviation value are compared with each other and the control signal is generated only if a sign of the first deviation value matches a sign of the second deviation value.

5. The method of claim 3, wherein an average value (Δ) is determined from the first deviation value and the second deviation value and the control signal is generated using the average value (Δ) as the at least one deviation value (Δ).

6. The method of claim 1, wherein an adjustment value is determined using the at least one deviation value (Δ) and an assignment rule, by which possible deviation values are each assigned an adjustment value; wherein an output value is determined using the first input value and the adjustment value; and
wherein the control signal is generated using the output value.

7. The method of claim 6, wherein an output value is determined using the first input value and the adjustment value by adding the first input value and the adjustment value

8. The method of claim 6, wherein the assignment rule is a sigmoid function or is based on a sigmoid function.

9. The method of claim 6,
wherein the assignment rule is defined as follows:
$$f ( Δ ) = \frac{2 \times S}{1 + {2}^{- Δ}} - S ,$$
where f(Δ) is the adjustment value, Δ is the at least one deviation value (Δ), and S is a maximum permissible magnitude(S) of the adjustment value.

10. The method of claim 9, wherein an approximation P for a term 2^{-Δ} based on a series expansion is determined and the assignment rule is defined as follows:
$$f ( Δ ) = \frac{2 \times S}{1 + P} - S .$$

11. The method of claim 10, wherein the approximation P for the term 2^{-Δ} is based on a Taylor series.

12. The method of claim 10, wherein the approximation P for the term 2^{-Δ} is based on a Maclaurin series.

13. The method of claim 10,
wherein the approximation P is determined as follows:
$$if - Δ \geq 0 , then P = 1 + {\sum }_{n = 1}^{N} \frac{{( k \times ( - Δ ) )}^{n}}{n !}$$
$$and / or$$
$$if - Δ < 0 , then P = \frac{1}{1 + {\sum }_{n = 1}^{N} \frac{{( - k \times ( - Δ ) )}^{n}}{n !}} .$$
where N is a predetermined order of the series expansion and k is a predetermined factor.

14. The method of claim 1, wherein a distance between the minimum target value (t) and the maximum target value (t+ε) is up to 10 nanoamperes and/or the minimum target value (t) is from 1 to 5 nanoamperes.

15. The method of claim 1, wherein a distance between the minimum target value (t) and the maximum target value (t+ε) is up to 5 nanoamperes and/or the minimum target value (t) is from 1 and 3 nanoamperes.

16. The method of claim 1, wherein a distance between the minimum target value (t) and the maximum target value (t+ε) is up to 1 nanoampere and/or the minimum target value (t) is 2 nanoamperes.

17. A control unit, wherein the control unit comprises elements configured for carrying out the method of claim 1.

18. A pulse oximeter, wherein the pulse oximeter comprises:
a light source;
a light sensor configured for converting a light component transmitted and/or reflected by a body part on irradiation with light from the light source into a sensor signal; and
the control unit of claim 17.

19. A computer program, wherein the program comprises commands which cause a processor to carry out the method of claim 1 when the processor executes the computer program.

20. A computer-readable medium on which the computer program of claim 19 is stored.
